(19) United States
(12) Patent Application Publication
(10) Pub. No.: US 2007/0255195 A1
(43) Pub. Date: Nov. 1, 2007

(54) Iontophoresis device

(76) Inventor: Hirotoshi Adachi, Tsukuba-shi (JP)

Correspondence Address: TOWNSEND & BANTA;c/o PORTFOLIO IP, PO BOX 52050, MINNEAPOLIS, MN, 55402, US

(21) Appl. No.: 11/659,908

(22) PCT Filed: Aug. 11, 2005
(86) PCT No.: PCT/JP05/14737

§ 371 Date: Feb. 8, 2007

(30) Foreign Application Priority Data
Aug. 11, 2004 (JP) 2004-234858

(51) Int. Cl. A61N 1/30

(52) U.S. Cl. 604/20

(57) ABSTRACT

An iontophoresis device which allows excellent transdermal absorption of an anionically chargeable physiologically active substance is provided.

The iontophoresis device comprises a cathode (25) and an interface (31) or (32) composed of a cationically chargeable membrane, wherein an anionically chargeable physiologically active substance (drug) (10) is placed between the cathode (25) and the interface (31) or in the interface (32). A wall member (13) with an adhesive layer (12) in the bottom is placed around an absorbent (11), and a support (15) having an opening (14) in the center is placed on the absorbent (11) and the wall member (13). When the device is used, the dissolution liquid for the drug is supplied to the absorbent (11) through the opening (14). The absorbent (11) and the interface (31) or (32) become wet with the dissolution liquid and the drug (10) is activated uniformly.

### TECHNICAL FIELD

The present invention relates to an iontophoresis device for transdermal administration of an anionically chargeable physiologically active substance.

### BACKGROUND ART

Iontophoresis is a method for delivering a drug from the skin or the mucous membrane by using electrical energy (e.g. Acta Dermatol Venereol., 64, 93, 1984). For performing such a method, an iontophoresis device with a desired configuration for delivering a physiologically active substance is used.

Conventionally, in an iontophoresis system, two electrodes connected to a power source, for example, placed in contact with the skin. The one electrode is called a donor electrode, from which a physiologically active substance (drug) is administered to the body. The other electrode is called a counter electrode, which is used for forming a closed circuit between the power source and the donor electrode. In such an iontophoresis system, when the physiologically active substance to be administered is cationically chargeableness which is positively chargeable and, an anode is the donor electrode, and a cathode is the counter electrode. On the contrary, when the physiologically active substance to be administered is anionically chargeableness which is relatively negatively chargeable, a cathode is the donor electrode, and the anode is the counter electrode.

In this type of device, for example, silver is used as a material for the anode, for example, and silver chloride is used as a material for the cathode, for example. Here, it should be noticed that a redox reaction occurs in these electrodes by an operation of the iontophoresis system, and as a result, another ion having a charge the same as in the drug ion is generated. The other ion is a competitive ion against the drug ion, and as a result, there is a problem that efficacy of administration of the drug by the iontophoresis is reduced. For example, when silver chloride is used as the cathode in transdermal administration of the anionically chargeable physiologically active substance, silver chloride is reduced to generate a chloride ion in the operation of the iontophoresis system. Since the chloride ion generated in this electrode complete with the anionically chargeable physiologically active substance, efficiency of transdermal administration of the anionically chargeable physiologically active substance is reduced.

For solving the above problem, Japanese Patent Laid-Open No. 9-511662 proposes device of a cation exchange substance layer. The cation exchange substance layer is placed between a cathode and a drug reservoir. An anion generated during electrode reduction is reacted with a cation in the cation exchange substance layer to form an electrically neutral or substantially insoluble compound, which aims to substantially exclude the competition of the anion generated during the electrode reduction with the anionic drug in a drug reservoir. Patent document 1: Japanese Patent Laid-Open No. 9-511662

### DISCLOSURE OF THE INVENTION

In the above-described prior art, although countermeasures for the drug ion against the generated competitive ion in the cathode are taken, no countermeasures for the cation delivered from the skin are taken. Since the cation competes with anionic drug ion administered transdermally, there is a problem to decrease delivery efficiency of the drug ion.

Consequently, an object of the present invention is to solve the problem in the prior art and to provide an iontophoresis device which allows excellent transdermal absorption of an anionically chargeable physiologically active substance.

The present inventors have extensively studied in order to solve the above problem and have found that an iontophoresis device which allows excellent transdermal absorption of an anionically chargeable physiologically active substance can be obtained by using a cationically chargeable interface. This finding has led to the completion of the present invention. Since the cation delivered from the skin can not pass through the cationically chargeable interface provided, it does not adversely affect the delivery efficiency of the anionic drug ion. Since the competitive ion does not exist in the cathode at the initial stage of electric supply to the iontophoresis, the anionic drug ion can be delivered to the skin without competition. Although the competitive ion (e.g. chloride ion in case of the silver chloride electrode) generated from the cathode is gradually increased and the delivery efficiency of the anionic drug ion is decreased, since inversion of the cation from the skin can be blocked by an action of the cationically chargeable interface, the transdermal absorption of the anionically chargeable physiologically active substance can be excellent in general. In addition, the skin physiology is less adversely affected because only a small amount of the cation flows to the device.

The above object can be achieved by an iontophoresis device which comprises a cathode, an interface composed of an cationically chargeable membrane, and an anionically chargeable physiologically active substance placed between the cathode and the interface or in the interface. The cationically chargeable membrane has a zeta potential of preferably −5 mV or more. The anionically chargeable physiologically active substance can be alprostadil or alprostadil alfadex. Disaccharide can be added as a stabilizer to the anionically chargeable physiologically active substance. Preferable examples of the disaccharide are sucrose and lactose.

Further, the iontophoresis device of the present invention comprises a cathode, an interface composed of a cationically chargeable membrane, an anionically chargeable physiologically active substance placed between the cathode and the interface or in the interface, and means for supplying dissolution liquid to the physiologically active substance. The means for supplying the dissolution liquid can be configured as a dissolution liquid reservoir opened by pressing. The dissolution liquid may contain glycerol.

According to the present invention, an iontophoresis device which allows excellent transdermal absorption of an anionically chargeable physiologically active substance can be obtained.

### BRIEF DESCRIPTION OF THE DRAWINGS

FIG. 1 is a cross-sectional view showing a configurational example of the iontophoresis device of the present invention, in which (a) a drug is placed between a cathode and an interface, or (b) a drug is placed in an interface;

FIG. 2 is a cross-sectional view showing another configurational example of the iontophoresis device of the present invention, in which (a) a drug is placed between a cathode and an interface, or (b) a drug is placed in an interface;

FIG. 3 is a graph showing the amount of cumulative permeation (μg/cm²) of alprostadil;

FIG. 4 is a graph showing the amount of cumulative permeation (μg/cm²) of lidocaine;

FIG. 5 is a graph showing the amount of cumulative permeation (μg/cm²) of alprostadil; and

FIG. 6 is a graph showing the blood concentration of PGE l(ng·eg/ml).

### DESCRIPTION OF SYMBOLS

10 Drug
11 Absorbent
12 Adhesive layer
13 Wall member
14 Opening
15 Support
25 Cathode
26 Lead part
31, 32 Interface

### BEST MODE FOR CARRYING OUT THE INVENTION

The iontophoresis device of the present invention (pharmaceutical preparation) comprises a cathode and an interface composed of a cationically chargeable membrane, wherein the anionically chargeable physiologically active substance (hereinafter referred to as drug) is placed between the cathode and the interface or in the interface.

FIG. 1 is a cross-sectional view showing a structure illustration of the iontophoresis device of the present invention, in which (a) a drug is placed between the cathode and the interface, or (b) a drug is placed in the interface.

The iontophoresis device shown in FIG. 1 (a) comprises a cathode 25 and an interface 31 composed of a cationically chargeable membrane, and wherein an absorbent 11 composed of a material, which contains a dry drug 10 and is able to absorb a liquid, is placed between the cathode 25 and the interface 31. A wall member 13 with an adhesive layer 12 in the bottom is placed around the absorbent 11, and a support 15 having an opening 14 in the center is placed on the absorbent 11 and the wall member 13. A lead part 26 for connection to an external power source is connected to the cathode 25. The interface 31 in FIG. 1(a) contains no drug. The iontophoresis device shown in FIG. 1(b) is different in that the drug 10 is not contained in the absorbent 11 but is contained in the interface 32, and others are the same as in FIG. 1(a).

When using any of the devices in FIG. 1(a) and (b), dissolution liquid is supplied by using a syringe (not shown) to the absorbent 11 through the opening 14 of the support 15. The absorbent 11 and the interface 31 or 32 become wet with the dissolution liquid and the drug 10 is activated uniformly. As a result, the drug penetrates into the skin through the interface by supplying power to the iontophoresis. Since the interface 31 or 32 is an interface composed of a cationically chargeable membrane and a cation delivered from the skin can not penetrate it, the drug can be administered through the skin efficiently.

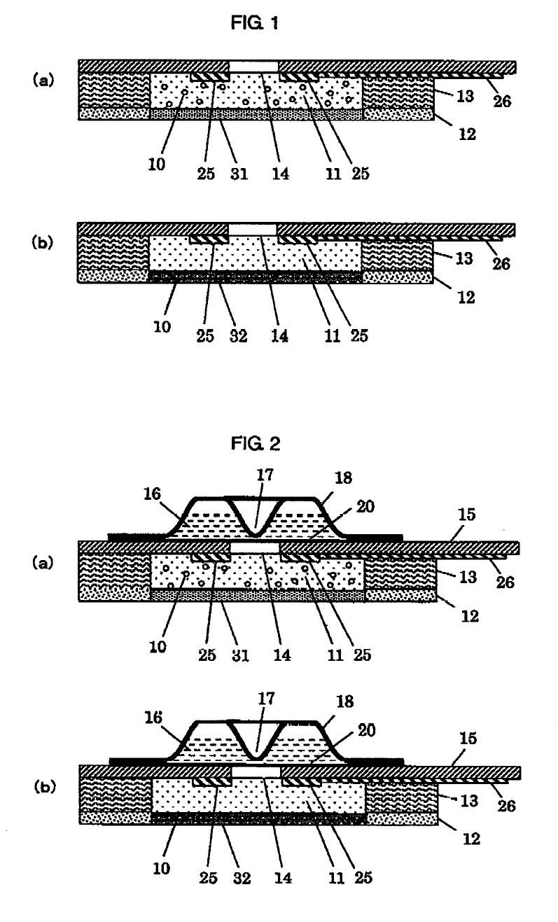

FIG. 2 shows a sectional view illustrating another configuration of the iontophoresis device of the present invention, in which (a) a drug is placed between a cathode and an interface, or (b) a drug is placed in an interface.

The iontophoresis device shown in FIG. 2(a) comprises a cathode 25 and an interface 31 composed of a cationically chargeable membrane, wherein an absorbent 11 composed of a material, which contains a dry drug 10 and is able to absorb a liquid, is placed between the cathode 25 and the interface 31. A wall member 13 with an adhesive layer 12 in the bottom is placed around the absorbent 11, and a support 15 having an opening 14 in the center is placed on the absorbent 11 and the wall member 13. A diaphragm 20 is placed on the support 15. On the diaphragm 20, a dissolution liquid reservoir 18 with a protruding part 17 for retaining dissolution liquid 16 for dissolving a drug within the area between the protruding part 17 and the diaphragm 20 and for destroying the diaphragm 20 by pressing is placed. The protruding part 17 has for instance a linear tip as shown in the Figure, which is arranged in contact with or adjacent to the diaphragm 20. A lead part 26 for connection to an external power source is connected to the cathode 25. The interface 31 in FIG. 2 (a) contains no drug. The iontophoresis device shown in FIG. 2 (b) is different in that the drug 10 is not contained in the absorbent 11 but is contained in the interface 32, and others are the same as in FIG. 2 (a).

When using any of the device in FIG. 2 (a) and (b), an upper plane of the liquid reservoir 18 is pressed to break the diaphragm 20 by the protruding part 17. In this occasion, the diaphragm 20 is broken along with the linear tip of the protruding part 17 to flow out the dissolution liquid 16 in the liquid reservoir 18 through the opening 14 of the support 15 into the absorbent 11. The absorbent 11 and the interface 31 or 32 become wet with the dissolution liquid and the drug 10 is activated uniformly. As a result, the drug penetrates into the skin through the interface by supplying power to the iontophoresis. Since the interface 31 or 32 is an interface composed of a cationically chargeable membrane and the cation delivered from the skin cannot penetrate it, the drug can be administered through the skin efficiently.

Zeta potential of the cationically chargeable membrane of the interface is about −7 mV or more, preferably about −5 mV or more under condition of the membrane in the dissolution liquid or after dissolution. In case of zeta potential below the above condition, the transdermal absorption of the drug deteriorates. The drug can be an anionically chargeable physiologically active substance, a part of which is at least dissociated to an anion, such as alprostadil or alprostadil alfadex. Stabilizers, drug dissolution rate adjusters, pH adjusters, absorption enhancers, etc. can be added. Disaccharide such as lactose, etc. can be added as the stabilizer. The drug and additives can be prepared by dissolving in the solvent such as water, an alcohol like ethanol, or a mixture thereof, which can dissolve the drug and additives therein, if necessary with heating to dissolve (dissolution process); dropping simultaneously or individually onto the cationically chargeable membrane (dropping process); and drying (drying process). To the above solution can be added an additive effective to make the membrane cationically chargeable or a substance for further improving drug absorption. Examples of such an additive are water, alcohols, polyalcohols. surfactants, sugars, pH adjusters, salts, water soluble polymers, solubilizing agents, absorption enhancers, oils and fats, and preservatives. Addition of glycerol as polyalcohol is preferable. The concentration of glycerol to be added is preferably 50% by weight or less. If the concentration of glycerol to be added exceeds 50% by weight, it is not preferable since voltage in electrification of iontophoresis is too high.

The power source for supplying electric energy to the iontophoresis device of the present invention is not specifically limited and is preferably an electric source which can apply a continuous or pulse direct current. Electric current of the continuous direct current is preferably 0.01 to 4 mA/cm², and voltage is preferably 2 to 20 V. In case of the pulse direct current, the frequency is preferably 0.1 to 200 KHz; the ratio of ON-OFF is preferably 1/100-20/1; and the electric current of the pulse direct current is preferably in a range of 0.01 to 4 mA/cm². Further, the voltage of pulse direct current is preferably 2 to 20 V.

In addition, the electrification condition can be set variously depending on the type and amount of the physiologically active substance to be administered.

The anionically chargeable physiologically active substance (drug) used in the present invention can be a physiologically active substance, and if at least a part thereof can be dissociated to an anion, all drugs in the medical field are included. Examples are curative, preventive medicines, anti-infectious disease drug such as antibiotics and antiviral drugs, analgesics, combined analgesics, anesthetics, anorectic drugs, anti-arthritic drugs, antiasthmatics, anticonvulsants, antidepressants, antidiabetics, antidiarrheal drugs, antihistamine, antiinflammatory agents, anti-migraine drugs, anti-motion sickness drugs, anti-vomiting drugs, antitumor agents, antiparkinson drugs, antipruritics, antipsychotics, antipyretics, antispasmodics for gastrointestine and urinary tract, anticholinergics, sympathomimetics, xanthine derivatives, drugs for cardiovascular system including calcium channel blockers, β-blockers, β-agonists, antiarrhythmics, antihypertensive drugs, ACE inhibitors, diuretics, systemic, coronary, peripheral and cerebral vasodilators, CNS stimulants, antitussive drugs, cold remedies, decongestants, diagnostic reagents, hormones, hypnotics, immunosuppressants, muscle relaxants, parasympatholytics, parasympathomimetics, prostaglandins, proteins, peptides, polypeptides, psychoanaleptics, sedatives, tranquilizers, etc.

Examples of the anionically chargeable physiologically active substance are; alprostadil, alprostadil alfadex, amoxicillin, ampicillin, aspoxicillin, benzyl penicillin, methicillin, piperacillin, sulbenicllin, ticarcillin, cefaclor, cefadroxil, cephalexin, cefatrizine, cefixime, cefradine, cefroxadine, cefamandole, cefazolin, cefmetazole, cefminox, cefoperazone, cefotaxime, cefotetan, cefoxitin, cefpiramide, cefsulodln, ceftazidime, ceftizoxima, ceftriaxone, cefuzonam, aztreonam, carumona, flomoxef, imipcncm, latamoxef, aiprofloxacin, enoxacin, nalidixic acid, norfloxacin, ofloxacin, vidarabine, fluorouracil, methotrexate, dexamethasone sodiumphosphate, levothyroxine, liothyronine, amlexanox, cromoglic acid, tranilast, gliclazide, insulins, benzbromarone, carbazochrome, tranexamic acid, alclofenac, aspirin, diclofenac, ibuprofen, ketoprofen, mefenamic acid, sulindac, tiaprofenic acid, tolmetin, sulpyrine, lobenzarit, penicillamine, amobarbital, pentobarbital, phenobarbital, thiopental, phenytoin, valproic acid, droxidopa, levodopa, baclofen, dantrolene, denopamine, furosemide, acetazolamide, bumetanide, canrenoic acid, etacrynic acid, alacepril, captopril, lisinopril, methyldopa, clofibrate, pravastatin, probucol, aminophylline, theophylline, carbocisteine and a salt thereof.

With regard to examples of the anionically chargeable physiologically active substance of the present invention, peptide, polypeptide and protein can be mentioned. These polymers have typically at least a molecular weight of about 300 dalton, more typically the molecular weight within a range of about 300-40,000 dalton. Generally, net charge on the polypeptide or protein can be retained in negative state (i.e. as anion) by retaining pH of the polypeptide/protein reservoir that is higher than the isoelectric point of the polypeptide or protein. Specific examples of peptides and proteins are not limited, and are LHRH. LHRH analog such as buserelin, gonadorelin, nafarelin, and leuprolide. GHRH, GHRF, insulin, insulinotropin, heparin, calcitonin, octreotide, endorphin, TRH, NT-36 (chemical name: N-[[(s)-4-oxo-2-azetidinil]carbonyl]-L-histidyl-L-prolinamide), lypressin, pituitary hormone such as HGH, HMG, HCG, desmopressin acetate, etc., follicular luteoid, αANF, growth factors such as growth factor releasing factor (GFRF), βMSH, somatostatin, bradykinin, somatotropin, platelet-derived growth factor, asparginase, bleomycin sulfate, chymopapain, cholecystokinin, chorionic gonadotropin, corticotropin (ACTH), erythropoietin, epoprostenol (platlet coagulation inhibitory factor), glucagons, hirulog, hyaluronidase, interferon, interleukin-1, interleukin-2, menotropins such as urofollitropin (FSH) and LH, oxytocin, streptokinase, tissue plasminogen activator, urokibase, vasopressin, desmopressin, ACTH analog, ANP, ANP clearance inhibitory factor, angiotensin II antagonist, antidiuretic hormone agonist, antidiuretic hormone antagonist, bradykinin antagonist, CD4, ceredase, CSF, enkephalin, FAB fragment, IgE peptide suppressing factor, IGF-1, neurotrophic factor, colony-stimulating factor, parathyroid hormone and agonist, parathyroid hormone antagonist, prostaglandin antagonist, protein C, protein S, renin inhibitory factor, thymosin α-1, thrombolytic drug, TNF, vaccine, vasopressin antagonist analog, α-1 anti-trypsin (recombinant) and TGF-β. These peptides, polypeptides and proteins are charged to negative depending on pH of the dissolution medium in higher PH than each isoelectric point.

Disaccharide used in the present invention acts as stabilizer for anionic physiologically active substance.

Disaccaride is not specifically limited and is sucrose (non-reductive), maltose (reductive), lactose (reductive), trehalose (non-reactive), cellobiose, isomaltose, etc. Among them, sucrose or lactose is preferable.

Examples of cationically chargeable membrane used in the present invention are, without limitation, preferably fine porous material which comprises polyolefin such as polyethylene, polyester such as PET, polycarbonate, poly(vinyl chloride), polyamide such as nylon, polyamide, polyacrylonitrile, polystyrene derivatives, ethylene-vinyl acetate copolymer, ethylene-poly(vinyl alcohol) copolymer. fluororesin, acrylic resin, epoxy resin, cellulose derivatives, and polysulfone such as PES. Further, in case that the physiologically active substance is protein etc., a membrane with low protein absorption is preferably used.

The thickness of cationically chargeable membrane is preferably 10 to 200 μm. The pore size of the membrane may be within a range in which the retention volume and releasability of the drug do not deteriorate, and drug is rapidly released from the membrane after bringing into contact with the dissolution liquid, thereby forming a highly concentrated drug solubility layer on the contact surface of the skin, and is 0.05 to 100 μm, preferably 0.1 to 10 μm, when taking into consideration efficiency in absorbability of the drug caused by charging condition of the membrane. Further, porosity of cationically chargeable membrane is preferably as high as possible within the range for retaining physical strength, preferably about 60 to 90%. In addition, it is better to be modified with an amino group or a secondary to quaternary ammonium group for charging.

The chargeable membrane may be a commercially available membrane, for example. Biodyne A, Biodyne B, Biodyne Plus (Japan Pall Corp.), High Bond N+ (Amersham Biosciences) and Immobilon Ny+ (Millipore Corp.).

Examples of the present invention will be explained hereinbelow. In examples and Comparative Examples, “%” means % by weight.

### TEST EXAMPLE 1

Effects of each interface on transdermal permeation of an anionically chargeable physiologically active substance were assessed by an in vitro permeation test using the skin of hairless mice.

Silver/silver chloride electrode was used as an electrode, and each interface was assembled in the contact face of the spacer layer (absorbent material) (nonwoven cloth) and the skin. An anionic physiologically active substance (alprostadil alfadex) (5 mg) dissolved in distilled water for injection (1.2 ml) was loaded in the spacer layer, end 0.2% sodium chloride solution was used as the receptor phase. An experimental procedure was performed in an air-bath adjusted to 32° C., wad the physiologically active substance in the receptor phase was measured in a time-dependent manner by using HPLC.

Power was supplied by a direct current at 1 mA for 2 hours (constant-current). Test Examples using Biodyne A and Biodyne B (Japan Pall Corp.) as the interface were referred to as “Example 1” and “Example 2” respectively. An Test Example using Biodyne C (Japan Pall Corp.) as the interface was referred to as “Comparative example 1”. An Test Example without the interface was referred to as “Comparative example 2”. Types and polarities of each interface are as shown in Table 1.

TABLE 1
Drug and each interface (types and polarities)

| | | | Membrane charge |
|---|---|---|---|
| | Drug | Interface | (pH4-6) |
| Example 1 | Alprostadil | Biodyne A | Positively and |
| | alfadex | | negatively charged* |
| Example 2 | Alprostadil | Biodyne B | Positively charged |
| | alfadex | | |
| Comparative | Alprostadil | Biodyne C | Negatively charged |
| Example 1 | alfadex | | |
| Comparative | Alprostadil | None | — |
| Example 2 | alfadex | | |

*Totally positively charged

Results of the in vitro transdermal permeation test in Test Example 1 are shown in FIG. 3.

When the cationically chargeable (positively charged) interface (Examples 1 and 2) was used, an anionic drug alprostadil alfadex was shown high drug permeation as compared with the case using an anionically chargeable (negatively charged) interface (Comparative Example 1) and the case without using the interface. Results demonstrated that the cationically chargeable interface promoted permeation of iontophoresis of the anionic physiologically active substance.

### TEST EXAMPLE 2

Similar to the Test Example 1, effects of each interface on transdermal permeation of a cationic physiologically active substance was assessed by an in vitro permeation test using the skin of hairless mice.

A silver electrode was used as an electrode. A cationic physiologically active substance (lidocaine hydrochloride 0.1%) dissolved in distilled water for injection was loaded in the spacer layer (absorption material, and power was supplied by a direct current at 1 mA for 2 hours (constant-current). In Comparative Example 3, Biodyne B (Japan Pall Corp.) was used as the interface. In Comparative Example 4, Biodyne C (Japan Pall Corp.) was used as the interface. In Comparative Example 5, no interface was used. Types and polarities of each interface are as shown in Table 1.

TABLE 2
Drug and each interface (types and polarities)

| | | | Membrane charge |
|---|---|---|---|
| | Drug | Interface | (pH4-6) |
| Comparative | Lidocaine | Biodyne B | Positively charged |
| Example 3 | hydrochloride | | |
| Comparative | Lidocaine | Biodyne C | Negatively charged |
| Example 4 | hydrochloride | | |
| Comparative | Lidocaine | None | — |
| Example 5 | hydrochloride | | |

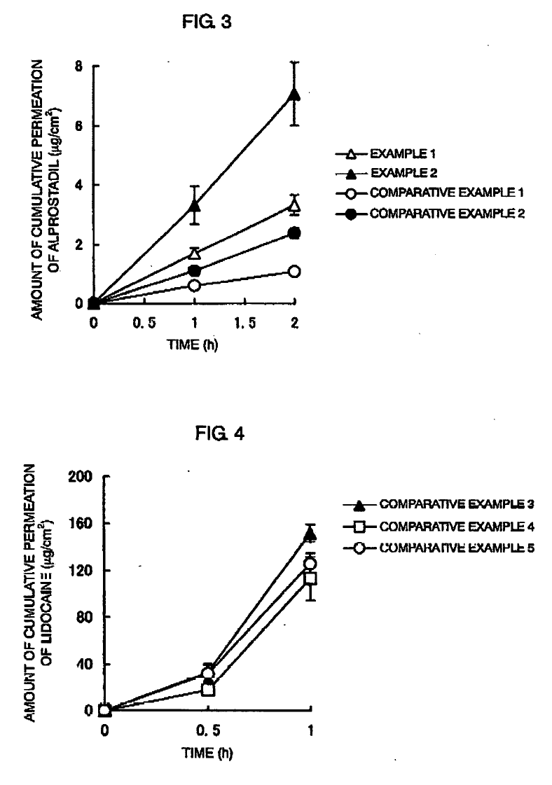

Results of Comparative Examples 3 to 5 are shown in FIG. 4. As a result of using lidocaine hydrochloride, a cationic physiologically active substance, even in case of using the cation chargeable interface (Comparative Example 3) and the anion chargeable interface (Comparative Example 4), almost the same skin permeation was obtained as compared with Comparative Example 5 without using the interface. Results demonstrated that in case of the cationic physiologically active substance, even if the anionically chargeable interface and the cationically chargeable interface were used or no interface was used, no effect on permeation of the physiologically active substance was found.

### TEST EXAMPLE 3

Effects of charge of each interface shown in Table 3 hereinbelow on transdermal permeation of alprostadil were assessed.

Zeta potential was measured by using a laser zeta electrometer (ELS-8000: Otsuka Electronics Co., Ltd.). Measurement was performed by using 10 mM NaCil (adjusted to pH 5 by adding hydrochloric acid) as a solvent under temperature at 25° C., electric field −32 V/cm. and calculated by Smoluchowski's equation under the condition in which the viscosity of solvent (η) was 0.881, the dielectric constant (ε) was 78.62, and the refraction index (n) was 1.331.

Under the same condition as in Test Example 1, effects of charging in each interface on transdermal permeation of alprostadil was assessed. Results are shown in Table 3.

TABLE 3
Zeta potential of each interface and transdermal
permeation of alprostadil

| | | | | Cumulative |
|---|---|---|---|---|
| | | | | amount of |
| | | | | permeation |
| | | Membrane | Zeta | of |
| | | charge | potential | alprostadil |
| | Interface | (pH4-6) | (mV, pH6) | (μg/cm²) |
| Example 3 | Biodyne B | Positively | +6.2 | 13.3 ± 0.6 |
| | | charge | | |
| Example 4 | Highbond N+ | Positively | +1.7 | 10.8 ± 0.4 |
| | | charge | | |
| Example 5 | Immobilon | Positively | +26.0 | 6.8 ± 0.1 |
| | Ny+ | charge | | |
| Example 6 | Highbond N | Positively | +10.4 | 6.7 ± 1.0 |
| | | and | | |
| | | negatively | | |
| | | charged* | | |
| Example 7 | Immobilon | Positively | −1.1 | 4.1 ± 0.5 |
| | Ny+ | and | | |
| | | negatively | | |
| | | charged* | | |
| Comparative | Durapore | No charge | −22.3 | 2.1 ± 0.1 |
| Example 6 | | | | |
| Comparative | CMF | No charge | −7.1 | 2.0 ± 0.2 |
| Example 7 | | | | |

*Totally positively charged

As shown in Table 3, the zeta potential of the interface differes depending on quality of material. This is caused by an interaction of an ionic functional group of the interface with the dissolution liquid as a medium. In case of a membrane having at least partially cationically chargeableness (positively charged) in the interface (Examples 3 to 7), the zeta potential shows a value of −1.1 mV or more. In this condition, transdermal permeation of alprostadil was significantly increased. Considering that the zeta potential value changes depending on the types of drug, the amount of transdermal permeation is significantly increased when zeta potential ranges −5.0 mV or more.

### TEST EXAMPLE 4

Effects of the position of each interface placed on transdermal permeation of alprostadil were assessed under same condition as in Test Example 1. Biodyne B (Japan Pall Corp.) was used as the interface; the interface was placed between the spacer and the skin in “Example 8”; the interface was placed between the spacer and the electrode in “Comparative Example 8”; the interface was placed halfway between the spacer and the spacer (halfway between the skin and the electrode) in “Comparative Example 9”; and no interface was used in “Comparative Example 10”. Results are shown in FIG. 5.

As shown in FIG. 5. in “Example 8”, in which the interface is placed in the contact face to the skin, showed higher transdermal permeation of alprostadil than in Comparative Examples 8, 9 and 10. Comparative Examples 8 and 9, in which the interface was not placed on the skin, showed almost equal to result of Comparative Example 10 without using the interface From these results, it was suggested that the place of arrangement of the interface in this pharmaceutical preparation is important to exhibit pharmaceutical ability.

### TEST EXAMPLE 5

Using pharmaceutical preparations shown in FIG. 1 (a), a drug (alprostadil alfadex 5 mg) was added and stored in an aluminum package. Pharmaceutical preparations were prepared as follows: no stabilizer was used in Comparative Example 11; the drug together with lactose (6 mg) was added in Example 9; a drying agent (Ozo 1 G, K. K. Sekkodo) was used in “Example 10”; and lactose (6 mg) together with a drying agent was used in Example 11. Prepared pharmaceutical preparations were stored at 50° C. for one month, and the content of alprostadil was measured by HPLC. In Table 4, the ratio of the remaining drug after stored at 50° C. for one month was shown as compared with the initial value.

TABLE 4
Effect of stabilizer on drug stability in
pharmaceutical preparation

| | | Remaining amount |
|---|---|---|
| | | of drug |
| | Stabilizer | (vs. initial value %) |
| Example 9 | Lactose | 78.4 ± 2.8 |
| Example 10 | Drying agent | 81.8 ± 2.1 |
| Example 11 | Lactose and drying | 95.8 ± 1.4 |
| | agent | |
| Comparative | No stabilizer | 38.9 ± 7.4 |
| Example 11 | | |

As obvious from the results shown in Table 4, drug stability was improved in Examples 9 to 11, in which lactose or a drying agent as a stabilizer was added, as compared with Comparative example 11 without adding stabilizer.

### TEST EXAMPLE 6

Pharmaceutical preparations as shown in Table 5 hereinbelow were prepared by the same way as in Test Example 5, and products were stored at 50° C. for one month, and stability of alprostadil was examined.

In Table 5, the amount of remaining drug after stored at 50° C. for one month was shown as a ratio to the initial value. The following stabilizers were used: disaccharide (sucrose (6 mg), lactose (8 mg)): a zeolite-based drying agent (Ozo, K. K. Sekkodo); a silica gel-based drying agent (Sorb-It, SUD-CHMIE GmbH): a clay-based drying agent (Desi Pak, SUD-CHMIE GmbH); or a molecular sieve-based drying agent (Tri-Sorb, SUD-CHMIE GmbH).

TABLE 5
Effect of stabilizer on drug stability in
pharmaceutical preparation

| | | Remaining |
|---|---|---|
| | | amount of drug |
| | | (vs. initial |
| | Stabilizer | value %) |
| Example 12 | Sucrose (disaccharide) and drying | 95.2 ± 0.6 |
| | agent (zeolite-based) | |
| Example 13 | Lactose (disaccharide) and drying | 96.5 ± 0.4 |
| | agent (zeolite based) | |
| Example 14 | Lactose (disaccharide) and drying | 93.6 ± 1.0 |
| | agent (silica gel-based) | |
| Example 15 | Lactose (disaccharide) and drying | 96.6 ± 0.9 |
| | agent (clay-based) | |
| Example 16 | Lactose (disaccharide) and drying | 96.7 ± 0.6 |
| | agent | |
| | (molecular sieve-based) | |
| Comparative | D-sorbitol (monosaccharide) and | 27.6 ± 3.0 |
| Example 12 | drying agent (zeolite-based) | |
| Comparative | No stabilizer | 38.9 ± 7.4 |
| Example 13 | | |

As obvious from the results shown in Table 5, although stability of the drug was improved in Examples 12 to 16, in which disaccharides or various drying agents as the stabilizer were added, as compared with the result of Comparative Example 13 without using stabilizer, stability was decreased in Comparative Example 12, in which monosaccharide, D-sorbitol, was added.

### TEST EXAMPLE 7

Effects of compositions of the dissolution liquid on transdermal permeation of alprostadil were examined by using Biodyne B (Japan Pall Corp.) as the interface under the same condition as in Test Example 1. A solution of alprostadil alfadex (5 mg) and glycerol (each concentration) in an aqueous sodium hydroxide solution (pH 6, 1.2 ml) was loaded in the spacer layer. Cumulative amount of permeation after 2 hours is shown in Table 6.

TABLE 6
Effect of composition of the dissolution liquid on
transdermal permeation of alprostadil

| | | Cumulative amount of permeation |
|---|---|---|
| | dissolution | of alprostadil |
| | liquid | (μg/cm²) |
| Example 17 | Glycerol 10% | 12.6 ± 0.3 |
| Example 18 | Glycerol 30% | 16.2 ± 0.6 |
| Example 19 | Glycerol 50% | 18.7 ± 2.7 |
| Comparative | Glycerol 0% | 9.6 ± 1.7 |
| Example 14 | | |

As shown in Table 6, the amount of permeation of alprostadil is increased depending upon increase in the amount of glycerol added. However, since voltage during electrification is increased depending upon the concentration of glycerol added and when the concentration of glycerol added is larger than 50% by weight, the voltage is significantly increased, and thus the concentration of added glycerol is preferably 50% by weight or less.

### TEST EXAMPLE 8

Using a pharmaceutical preparation as shown in FIG. 1 (a), a preparation containing alprostadil alfadex (5 mg) and ³H-prostaglandin E₁ (³H-PGE₁, 3 μCi) as a drug was prepared. SD rats, male (body weight about 250 g) were anesthetized with 25% urethane (5 ml/kg). Abdominal skin was shaven by using hair clippers and a shaver and the skin surface was sterilized using alcohol. The above prepared pharmaceutical preparation was patched and fixed on the skin surface, and the pharmaceutical preparation was prepared by adding each dissolution liquid into the pharmaceutical preparation. The dissolution liquid used was as follows: a 30% glycerol solution in Example 20; and purified water without glycerol in Comparative Example 15. Power was supplied at 0.4 mA/cm² for 60 minutes, and blood was collected from the jugular vein in a time-dependent manner. Plasma was separated after centrifugation and radioactivity was measured by a liquid scintillation counter.

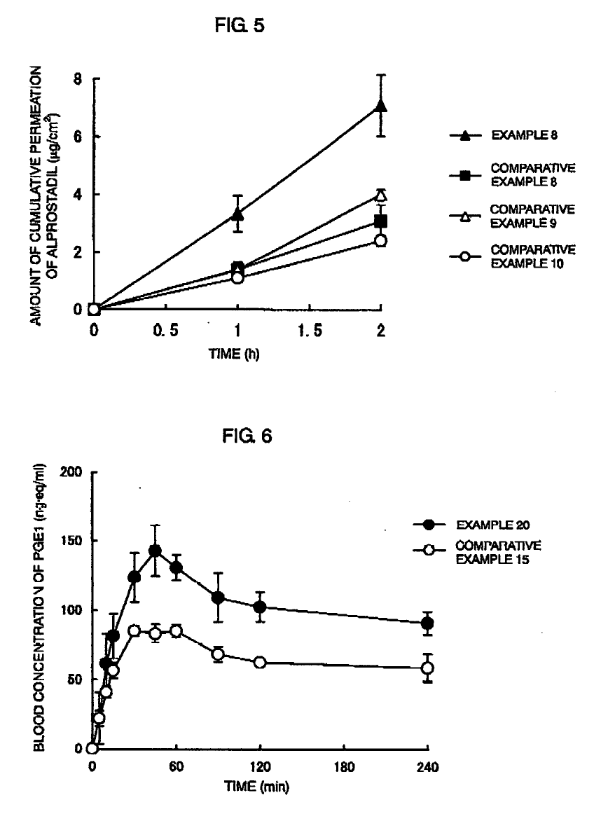

FIG. 6 shows changes of the concentration of plasma PGE₁, of each pharmaceutical preparation. Similar to results of the in vitro transdermal permeation test of hairless mice, a significant increase of absorption was observed in the experiment using the dissolution liquid containing 30% glycerol (Example 20) as compared with the experiment without addition of glycerol.

### TEST EXAMPLE 9

As shown in Table 7, in Examples 21 to 23 and Comparative Example 16, each dissolution liquid was loaded into the dissolution liquid reservoir for a pharmaceutical preparation shown in FIG. 2(a) (blister part) (made of PVC), and was heat bonded by an aluminum covering material. The thus prepared dissolution liquid container was packed in an aluminum package together with a drying agent (Ozo 1 G, K. K. Sekkodo) and stored at 50° C. for one month, then changes of weight were examined. In Table 7, weight loss during storage was shown in a rate of change from the initial weight.

TABLE 7
Weight changes during storage of dissolution liquid

| | dissolution | Weight loss |
|---|---|---|
| | liquid | (vs. initial value %) |
| Example 21 | Glycerol 10% | 5.7 |
| Example 22 | Glycerol 30% | 5.4 |
| Example 23 | Glycerol 50% | 4.8 |
| Comparative Example 16 | Glycerol 0% | 6.0 |

As shown in Table 7, addition of glycerol shows a tendency to suppress weight changes of physiologically active substance during storage.

### INDUSTRIAL APPLICABILITY

The present invention relates to an iontophoresis device for transdermal administration of an anionically chargeable physiologically active substance, and is industrially applicable.

1. An iontophoresis device comprising a cathode, an interface composed of a cationically chargeable membrane and an anionically chargeable physiologically active substance placed between the cathode and the interface or in the interface.

2. The iontophoresis device according to claim 1, wherein the cationically chargeable membrane has a zeta potential of −5 mV or more.

3. The iontophoresis device according to claim 1, wherein the anionically chargeable physiologically active substance is alprostadil or alprostadil alfadex.

4. The iontophoresis device according to claim 1, wherein disaccharide as a stabilizer is added to the anionically chargeable physiologically active substance.

5. The iontophoresis device according to claim 4, wherein the disaccharide is sucrose or lactose.

6. An iontophoresis device comprising a cathode, an interface composed of a cationically chargeable membrane, anionically chargeable physiologically active substance placed between the cathode and the interface or in the interface, and a unit supplying dissolution liquid to the physiologically active substance.

7. The iontophoresis device according to claim 6, wherein the unit supplying dissolution liquid is a dissolution liquid reservoir which is opened by pressing.

8. The iontophoresis device according to claim 6, wherein the dissolution liquid contains glycerol.

9. The iontophoresis device according to claim 2, wherein the anionically chargeable physiologically active substance is alprostadil or alprostadil alfadex.

10. The iontophoresis device according to claim 2, wherein disaccharide as a stabilizer is added to the anionically chargeable physiologically active substance.

11. The iontophoresis device according to claim 3, wherein disaccharide as a stabilizer is added to the anionically chargeable physiologically active substance.

12. The iontophoresis device according to claim 7, wherein the dissolution liquid contains glycerol.
